(19) United States
(12) Patent Application Publication
(10) Pub. No.: US 2015/0252013 A1
(43) Pub. Date: Sep. 10, 2015

(54) TREATMENT OF MULTIPLE SCLEROSIS AND PSORIASIS USING PRODRUGS OF METHYL HYDROGEN FUMARATE

(71) Applicant: XenoPort, Inc., Santa Clara, CA (US)

(72) Inventors: Peter A. Virsik, Portola Valley, CA (US); David J. Wustrow, Los Gatos, CA (US); Thamil Annamalai, Saratoga, CA (US); Suresh K. Manthati, Sunnyvale, CA (US)

(21) Appl. No.: 14/717,639

(22) Filed: May 20, 2015

Related U.S. Application Data
(63) Continuation of application No. 13/906,155, filed on May 30, 2013.
(60) Provisional application No. 61/653,375, filed on May 30, 2012.

(51) Int. Cl. C07D 295/185; C07D 207/08; C07C 235/14; C07C 235/06; C07D 295/088

(52) U.S. Cl. CPC C07D 295/185; C07C 235/06; C07D 295/088; C07C 235/14; C07D 207/08

(57) ABSTRACT

Improved methods of treating multiple sclerosis and/or psoriasis using prodrugs of methyl hydrogen fumarate are disclosed. The methods comprise administering certain prodrugs of methyl hydrogen fumarate. The methods are able to achieve high blood plasma concentrations of the active metabolite, methyl hydrogen fumarate, without causing significant gastrointestinal irritation. New prodrugs of methyl hydrogen fumarate are also disclosed.

This application is a continuation of U.S. patent application Ser. No. 13/906,155, filed May 30, 2013, which claims the benefit under 35 U.S.C. §119(e) of U.S. Provisional Application Ser. No. 61/653,375 filed May 30, 2012, the contents of which are incorporated by reference in their entirety.

### TECHNICAL FIELD

The present disclosure relates to methods of treating multiple sclerosis and/or psoriasis using prodrugs of methyl hydrogen fumarate (MHF) which achieve high therapeutic blood plasma concentrations of MHF in patients while avoiding serious gastrointestinal irritation and related side-effects.

### BACKGROUND

Fumaric acid esters, i.e., dimethylfumarate in combination with salts of ethylhydrogenfumarate, have been used in the treatment of psoriasis for many years. The combination product, marketed under the tradename Fumaderm®, is in the form of oral tablets and is available in two different dosage strengths (Fumaderm® initial and Fumaderm®):

| | Fumaderm ® | Fumaderm ® |
|---|---|---|
| Fumarate Compound | Initial (mg) | (mg) |
| Dimethylfumarate | 30 | 120 |
| Ethyl hydrogen fumarate, calcium salt | 67 | 87 |
| Ethyl hydrogen fumarate, magnesium salt | 5 | 5 |
| Ethyl hydrogen fumarate, zinc salt | 3 | 3 |

The two strengths are intended to be applied in an individually based dosing regimen starting with Fumaderm® initial in an escalating dose, and then after, e.g., three weeks of treatment, switching to Fumaderm®. Both Fumaderm® initial and Fumaderm® are enteric coated tablets.

Another marketed composition is Fumaraat 120 containing 120 mg of dimethyl fumarate and 95 mg of calcium monoethyl fumarate (TioFarma, Oud-Beijerland, Netherlands). The pharmacokinetic profile of Fumaraat 120® in healthy subjects is described in Litjens et al., Br. J. Clin. Pharmacol., 2004, vol. 58:4, pp. 429-432. The results show that a single oral dose of Fumaraat 120® is followed by a rise in serum monomethyl fumarate concentration and only negligible concentrations of dimethyl fumarate and fumaric acid is observed.

U.S. Pat. Nos. 6,277,882 and 6,355,676 disclose respectively the use of alkyl hydrogen fumarates and the use of certain fumaric acid monoalkyl ester salts for preparing microtablets for treating psoriasis, psoriatic arthritis, neurodermatitis and enteritis regionalis Crohn. U.S. Pat. No. 6,509,376 discloses the use of certain dialkyl fumarates for the preparation of pharmaceutical preparations for use in transplantation medicine or the therapy of autoimmune diseases in the form of microtablets or micropellets. U.S. Pat. No. 4,959,389 discloses compositions containing different salts of fumaric acid monoalkyl esters alone or in combination with dialkyl fumarate. GB 1,153,927 relates to medical compositions comprising dimethyl maleic anhydride, dimethyl maleate and/or a dimethyl fumarate.

However, oral administration of fumarates such as Fumaderm® frequently causes irritation of the gastric and intestinal tissues, which in turn causes fullness, diarrhea, upper abdominal cramps, flatulence and/or nausea.

Oral administration of dimethyl fumarate (DMF) has been in human clinical testing for the treatment of multiple sclerosis and has shown promising results in reducing multiple sclerosis relapses and MS disability progression. DMF is thought to be a prodrug of methyl hydrogen fumarate. Unfortunately, DMF is highly irritating to the skin and mucosal membranes with the result that oral administration of DMF tends to cause serious digestive tract irritation with attendant nausea, vomiting, abdominal pain and diarrhea. These serious side effects of oral administration of DMF limits the utility of this drug for treating diseases such as psoriasis and multiple sclerosis.

More recently, MHF prodrugs are disclosed in Gangakhedkar et al. U.S. Pat. No. 8,148,414 and Cundy et al. U.S. Patent Application 61/595,835 filed Feb. 7, 2012. Both of these disclose MHF prodrugs and their use for treating a number of medical conditions, including multiple sclerosis and psoriasis.

### SUMMARY

Disclosed herein are improved methods of treating multiple sclerosis and/or psoriasis in human patients using fumaric acid esters. The methods are able to achieve high therapeutic levels of MHF in the blood plasma of a patient without causing significant gastrointestinal irritation.

In one aspect, the treatment comprises orally administering one or more methyl hydrogen fumarate (MHF) prodrugs to a patient in need of such treatment.

The MHF prodrug(s) exhibits any one, or a combination of, the following effects and characteristics.

In some aspects, the MHF prodrug(s) exhibits an average gastrointestinal irritation score in an Annamalai-Ma gastrointestinal irritation rat model of no more than 3 after orally administering a solution or suspension of the prodrug to rats at a dose of 180 mg-equivalents of MHF per kg of body weight, dosed once per day over 4 consecutive days.

In some aspects, the MHF prodrug(s) exhibits a relative GST enzyme activity (GSTAᵣₑₗ) of less than 80%, where GSTA_{rd} is calculated in accordance with equation (I):

GSTAᵣₑₗ(%)=(SAR_{prodrug}÷SAR_{DMF})×100 (I)

wherein:

SAR_{prodrug} is the specific activity ratio of the MHF prodrug, and
SAR_{DMF} is the specific activity ratio of dimethyl fumarate.
In some aspects, the MHF prodrug(s) exhibits a human in vivo metabolism to MHF over 24 hours sufficient to achieve a ratio of AUC_{MMF 0-24}: AUC_{Prodrug 0-24} of at least 3:1.

In some aspects, the oral administration of the MHF prodrug(s) is sufficient to obtain (i) a therapeutic concentration of MHF in blood plasma of the patient of at least 0.5 μg/ml at a time within 24 hours after said oral administration; and (ii) an area under a concentration of MHF in blood plasma versus time curve (AUC) of at least 4.8 μg·hr/ml over 24 hours after start of the oral administration.

In another aspect, the treatment comprises orally administering one or more methyl hydrogen fumarate (MHF) prodrugs to a patient in need of such treatment. The MHF prodrug(s) exhibits a relative GST enzyme activity (GSTAᵣₑₗ) of less than 50%, where GSTA_{rd} is calculated in accordance with equation (I):

GSTAᵣₑₗ(%)=(SAR_{prodrug}÷SAR_{DMF})×100 (I)

wherein:

SAR_{prodrug} is the specific activity ratio of the MHF prodrug, and

SAR_{DMF} is the specific activity ratio of dimethyl fumarate.

In another aspect, the MHF prodrug(s) exhibits a human in vivo metabolism to MHF over 24 hours sufficient to achieve a ratio of AUC_{MMF 0-24} AUC_{Prodrug 0-24} of at least 9:1.

In some aspects, the oral administration of the MHF prodrug(s) is sufficient to obtain (i) a therapeutic concentration of MHF in blood plasma of the patient of at least 0.7 μg/ml at a time within 24 hours after said oral administration; and (ii) an area under a concentration of MHF in blood plasma versus time curve (AUC) of at least 7.0 μg·hr/ml over 24 hours after start of the oral administration.

In other aspects, the MHF prodrug(s) exhibits an average gastrointestinal irritation score in an Annamalai-Ma gastrointestinal irritation rat model of no more than 2 after orally administering a solution or suspension the prodrug to rats at a dose of 180 mg-equivalents of MHF per kg of body weight, dosed once per day over 4 consecutive days

In other aspects, the MHF prodrug(s) exhibits a human in vivo metabolism to MHF over 24 hours sufficient to achieve a ratio of AUC_{MMF 0-24}. AUC_{Prodrug 0-24} of at least 19:1.

The oral administration of the MHF prodrug(s) can be sufficient to obtain (i) a therapeutic concentration of MHF in blood plasma of the patient of at least 1.0 μg/ml at a time within 24 hours after said oral administration; and (ii) an area under a concentration of MHF in blood plasma versus time curve (AUC) of at least 12.0 μg·hr/ml over 24 hours after start of the oral administration.

In a another aspect, the treatment comprises administering an MHF prodrug of Formula (I), which MHF prodrug has the relative GST enzyme activity, in vivo metabolism and gastrointestinal irritation scores described above:

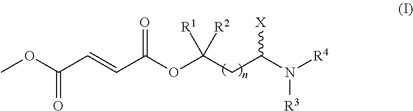

or a pharmaceutically acceptable salt thereof, wherein:

R¹ and R² are independently chosen from hydrogen, C₁₋₆ alkyl, and substituted C₁₋₆ alkyl;

R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₄₋₁₀ heteroaryl, substituted C₄₋₁₀ heteroaryl, C₄₋₁₀ heterocycloalkyl, and substituted C₄₋₁₀ heterocycloalkyl;

n is an integer from 0 to 4; and

X is independently chosen from a single oxygen atom and a pair of hydrogen atoms;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl;

and wherein when X is a single oxygen atom, the oxygen atom is connected to the carbon to which it is bonded by a double bond to form a carboxyl group and when X is a pair of hydrogen atoms, each hydrogen atom is connected to the carbon to which it is bonded to by single bond.

Also disclosed herein are prodrugs of methyl hydrogen fumarate, namely, (N-cyclopropyl-N-ethylcarbamoyl)methyl methyl 2(E)but-2-ene-1,4-dioate, (N-cyclopropyl-N-methylcarbamoyl)methyl methyl 2(E)but-2-ene-1,4-dioate, Methyl 2-oxo-2-pyrrolidinylethyl 2(E)but-2-ene-1,4-dioate, and pharmaceutically acceptable salts thereof.

Further disclosed are pharmaceutical compositions comprising a methyl hydrogen fumarate prodrug and a pharmaceutically acceptable vehicle.

In some embodiments, the pharmaceutical composition is an oral formulation.

In other embodiments, the pharmaceutical composition comprises a therapeutically effective amount of the methyl hydrogen fumarate prodrug for the treatment of a disease selected from multiple sclerosis and psoriasis.

### DETAILED DESCRIPTION

### Definitions

A dash (“-”) that is not between two letters or symbols is used to indicate a point of attachment for a moiety or substituent. For example, —CONH₂ is bonded through the carbon atom.

“Alkyl” refers to a saturated or unsaturated, branched, cyclic, or straight-chain, monovalent hydrocarbon radical derived by the removal of one hydrogen atom from a single carbon atom of a parent alkane, alkene, or alkyne. Examples of alkyl groups include, for example, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, cyclopropyl, cyclobutyl, cyclopentyl, etc.; and the like.

The term “alkyl” includes groups having any degree or level of saturation, i.e., groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl, or alkynyl are used. The term “alkyl” includes cycloalkyl and cycloalkylalkyl groups. In certain embodiments, an alkyl group can have from 1 to 10 carbon atoms (C₁₋₁₀), in certain embodiments, from 1 to 6 carbon atoms (C₁₋₆), in certain embodiments from 1 to 4 carbon atoms (C₁₋₄), in certain embodiments, from 1 to 3 carbon atoms (C₁₋₃), and in certain embodiments, from 1 to 2 carbon atoms (C₁₋₂). In certain embodiments, alkyl is methyl, in certain embodiments, ethyl, and in certain embodiments, n-propyl or isopropyl.

“Arylalkyl” refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀. In certain embodiments, an arylalkyl group is C₇₋₁₂ arylalkyl.

“AUC” refers to the area under a curve on which time is plotted on the X-axis and concentration of a substance (e.g., MHF) in blood or blood plasma is plotted on the Y-axis over a particular period of time (e.g., time zero to 24 hours). AUC is commonly expressed in units of mg·hr/ml.

“Compounds” of Formula (I) disclosed herein include any specific compounds within this formula. Compounds may be identified either by their chemical structure and/or chemical name. Compounds are named using Chemistry 4-D Draw Pro, version 7.01c (ChemInnovation Software, Inc., San Diego, Calif.). When the chemical structure and chemical name conflict, the chemical structure is determinative of the identity of the compound. The compounds described herein may comprise one or more chiral centers and/or double bonds and therefore may exist as stereoisomers such as double bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to those skilled in the art. Compounds of Formula (I) include, for example, optical isomers of compounds of Formula (I), racemates thereof, and other mixtures thereof. In such embodiments, a single enantiomer or diastereomer, i.e., optically active form can be obtained by asymmetric synthesis or by resolution of the racemates. Resolution of the racemates may be accomplished, for example, by methods such as crystallization in the presence of a resolving agent, or chromatography using, for example, chiral stationary phases. Notwithstanding the foregoing, in compounds of Formula (I) the configuration of the illustrated double bond is only in the E configuration (i.e., trans configuration).

Compounds of Formula (I) also include isotopically labeled compounds where one or more atoms have an atomic mass different from the atomic mass conventionally found in nature. Examples of isotopes that may be incorporated into the compounds disclosed herein include, for example, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N oxides. In general, compounds disclosed herein may be free acid, hydrated, solvated, or N oxides. Certain compounds may exist in multiple crystalline, co-crystalline, or amorphous forms. Compounds of Formula (I) include pharmaceutically acceptable salts thereof or pharmaceutically acceptable solvates of the free acid form of any of the foregoing, as well as crystalline forms of any of the foregoing.

Compounds of Formula (I) also include solvates. A solvate refers to a molecular complex of a compound with one or more solvent molecules in a stoichiometric or non-stoichiometric amount. Such solvent molecules include those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules are water.

Further, when partial structures of the compounds are illustrated, an asterisk (□*) indicates the point of attachment of the partial structure to the rest of the molecule.

“Cycloalkyl” refers to a saturated or partially unsaturated cyclic alkyl radical. Where a specific level of saturation is intended, the nomenclature cycloalkanyl or cycloalkenyl is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₁₋₆ cycloalkyl.

“Cycloalkylalkyl” refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀, and in certain embodiments, a cycloalkylalkyl group is C₃₋₂₀ cycloalkylalkyl, e.g., the alkanyl, alkenyl, or alkynyl moiety of the cycloalkylalkyl group is C₁₋₈ and the cycloalkyl moiety is C₃₋₁₂. In certain embodiments, a cycloalkylalkyl group is C₄₋₁₂ cycloalkylalkyl.

“Disease” refers to a disease, disorder, condition, or symptom of any of the foregoing.

“Drug” as defined under 21 U.S.C. §321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . . ”

“GST” and “GSTs” each refers to glutathione S-transferase enzymes.

“Heteroalkyl” by itself or as part of another substituent refer to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, —NR¹³, ═N—N═, —N═N—, —N═N—NR¹³—, —PR¹³—, —P(O)₂—, —POR¹³—, —O—P(O)₂, —SO—, —SO₂—, —Sn(R¹³)₂—, and the like, where each R¹³ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C_{7˜}heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R¹³ is independently chosen from hydrogen and C₁₋₃ alkyl. In certain embodiments, a heteroatomic group is chosen from —O—, —S—, —NH—, —N(CH₃)—, and —SO₂—; and in certain embodiments, the heteroatomic group is —O—.

“Heteroaryl” refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

“Heterocycloalkyl” refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, a heterocycloalkyl group is C₄₋₁₀ heterocycloalkyl, C₄₋₈ heterocycloalkyl, and in certain embodiments, C₄₋₆ heterocycloalkyl.

“Leaving group” has the meaning conventionally associated with it in synthetic organic chemistry, i.e., an atom or a group capable of being displaced by a nucleophile and includes halogen such as chloro, bromo, fluoro, and iodo; acyloxy, such as acetoxy and benzoyloxy, alkoxycarbonylaryloxycarbonyl, mesyloxy, tosyloxy, and trifluoromethanesulfonyloxy; aryloxy such as 2,4-dinitrophenoxy, methoxy, N,O-dimethylhydroxylamino, p-nitrophenolate, imidazolyl, and the like.

“MHF” refers to methyl hydrogen fumarate, a compound having the following chemical structure:

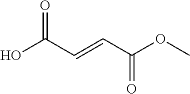

This compound is also sometimes referred to as monomethyl fumarate.

“MHF Prodrug” refers to a prodrug that is metabolized in vivo to form methyl hydrogen fumarate as a pharmacologically active metabolite.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of out-of-plane π-electrons corresponding to the Hückel rule (4n+2). Examples of heteroatoms to replace the carbon atoms include, for example, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, for example, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound that possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N methylglucamine, and the like. In certain embodiments, a pharmaceutically acceptable salt is the hydrochloride salt. In certain embodiments, a pharmaceutically acceptable salt is the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient, which does not destroy the pharmacological activity thereof and which is non-toxic when administered in doses sufficient to provide a therapeutically effective amount of the compound or a pharmacologically active metabolite thereof.

“Pharmaceutical composition” refers to a compound of Formula (I), or a pharmaceutically acceptable salt thereof, and at least one pharmaceutically acceptable vehicle, with which the compound of Formula (I), or a pharmaceutically acceptable salt thereof, is administered to a patient.

“Prodrug” refers to a compound administered in a pharmacologically inactive (or significantly less active) form. Once administered, the compound is metabolized in vivo into an active metabolite. Prodrugs may be designed to improve oral bioavailability, particularly in cases where the metabolite exhibits poor absorption from the gastrointestinal tract. Prodrugs can be used to optimize the absorption, distribution, metabolism, and excretion (ADME) of the active metabolite.

“Relative GST enzyme activity” and “GSTA_{rd}” refers to the quotient of the specific activity ratio of a test MHF prodrug divided by the specific activity ratio of dimethyl fumarate. Relative GST enzyme activity can be expressed as a percent in accordance with the following equation (I):

GSTAᵣₑₗ(%)=(SAR_{prodrug}÷SAR_{DMF})×100 (I)

“Specific activity ratio of an MHF prodrug” and “SAR_{prodrug}” each refers to the ratio of (i) the GST activity in the forestomach tissue of rats treated with the MMF prodrug to (ii) the GST activity in the forestomach tissue of rats treated with a non-irritating control solution comprised of 0.5 wt % methylcellulose and 0.1 wt % Tween 80 in 10 mM sodium acetate buffer, pH 4.5. The SAR_{prodrug} is proportional to the amount of GST isozymes induced by an MHF prodrug treatment.

“Specific activity ratio of DMF” and “SAR_{DMF}” each refers to the ratio of (i) the GST activity in the forestomach tissue of rats treated with DMF to (ii) the GST activity in the forestomach tissue of rats treated with a non-irritating control solution comprised of 0.5 wt % methylcellulose and 0.1 wt % Tween 80 in 10 mM sodium acetate buffer, pH 4.5. SAR_{DMF} is proportional to the amount of GST isozymes induced by DMF treatment.

“Substituent” refers to a group in which one or more hydrogen atoms are independently replaced (or substituted) with the same or substituent group(s). In certain embodiments, each substituent group is independently chosen from halogen, —OH, —ON, —CF₃, ═O, —NO₂, benzyl, —C(O)NH₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from halogen, —OH, —ON, —CF₃, —NO₂, benzyl, —R¹¹, —OR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, ═O, —NO₂, benzyl, —C(O)NR¹¹₂—, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from —OH, C₁₋₄ alkyl, and —NH₂.

“Treating” or “treatment” of any disease refers to reversing, alleviating, arresting, or ameliorating a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treating” or “treatment” also refers to inhibiting a disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of a disease or at least one or more symptoms thereof in a patient who may be exposed to or predisposed to a disease even though that patient does not yet experience or display symptoms of the disease.

“Therapeutically effective amount” refers to the amount of a compound that, when administered to a subject for treating a disease, or at least one of the clinical symptoms of a disease, is sufficient to effect such treatment of the disease or symptom thereof. The “therapeutically effective amount” may vary depending, for example, on the compound, the disease and/or symptoms of the disease, severity of the disease and/or symptoms of the disease, the age, weight, and/or health of the patient to be treated, and the judgment of the prescribing physician. An appropriate amount in any given compound may be ascertained by those skilled in the art and/or is capable of determination by routine experimentation.

“Therapeutically effective dose” refers to a dose that provides effective treatment of a disease in a patient. A therapeutically effective dose may vary from compound to compound and/or from patient to patient, and may depend upon factors such as the condition of the patient and the route of delivery. A therapeutically effective dose may be determined in accordance with routine pharmacological procedures known to those skilled in the art.

Reference is now made in detail to certain embodiments of compounds, compositions, and methods. The disclosed embodiments are not intended to be limiting of the claims. To the contrary, the claims are intended to cover all alternatives, modifications, and equivalents.

### Treatment Methods

The methods disclosed herein involve selecting a prodrug of methyl hydrogen fumarate having the appropriate physico-chemical characteristics and in vivo effects.

### GST Enzyme Activity

One in vivo effect of an MHF prodrug suitable for use in the presently described methods is a lower glutathione S-transferase (GST) activity level in rat forestomach tissue compared to equivalent oral dosing of dimethyl fumarate. GSTs make up a large family of Phase II detoxification enzymes that are broadly expressed in species ranging from bacteria to human with over 100 isoforms of this enzyme having been identified. (Buetler, T. M. et al., 1992, Environ. Carcinogen Eco-lox., Revs C10(2), 181-203.) Cytosolic GSTs expressed by mice, rats and humans are classified into three groups or families; alpha, mu and pi. (Mannervik, G., Proc. Natl. Acad. Sci., 1985, 7202-7206). These enzymes function to transfer the endogenous nucelophile gluthathione (GSH) to electrophilic compounds as part of a detoxifying mechanism for xenobiotics (Tsuchida, S. et al., Crit. Rec. Biochem. Molec. Biol., 1992, 27, 337-384). A variety of electrophilic agents including dimethyl fumarate have been shown to induce the expression of this enzymatic activity (Talalay, P. et al., Proc. Natl. Acad. Sci., 1988, 85, 8261-8265). Oral administration of DMF has been shown to cause tissue damage in the rat forestomach as evidenced by the observation of pachydermia, hyperplasia and hyperkeratosis (Fumaderm SPC 2009). GST activity in forestomach tissue is highly increased in rats receiving oral DMF (Spencer, S. R. et. al., Cancer Res., 1990, 50, 7871-7875). These data indicate that induction of GST activity is a marker of gastric irritation by fumaric acid esters. Since DMF is also an MMF prodrug (i.e., MHF is the circulating active moiety after oral administration of DMF), methods of orally administering an MMF prodrug that achieve similar or greater concentrations of MMF in the blood plasma compared to DMF oral administration, while causing less of an increase in GST activity is predictive of lowering gastrointestinal irritation compared to equivalent DMF dosing.

In accordance with the present methods, the level of GST enzyme activity induced by a particular MMF prodrug treatment can be compared to that induced by DMF treatment by dividing the specific activity ratio of the particular MMF prodrug (SAR_{prodrug}) by the specific activity ratio of DMF (SAR_{DMF}) to calculate a relative GST enzyme activity (GSTAᵣₑₗ) which can be expressed as a percent in equation (I):

GSTAᵣₑₗ(%)=(SAR_{prodrug}÷SAR_{DMF})×100 (I)

wherein SAR_{prodrug} is the ratio of (i) the GST activity in the forestomach tissue of rats treated with the MMF prodrug (GSTA_{prodrug}) to (ii) the GST activity in the forestomach tissue of rats treated with a non-irritating control solution comprised of 0.5 wt % methylcellulose and 0.1 wt % Tween 80 in 10 mM sodium acetate buffer, pH 4.5 (GSTA_{control}); and SAR_{DMF} is the ratio of (i) the GST activity in the forestomach tissue of rats treated with DMF (GSTA_{DMF}) to (ii) the GST activity in the forestomach tissue of rats treated with a non-irritating control solution comprised of 0.5 wt % methylcellulose and 0.1 wt % Tween 80 in 10 mM sodium acetate buffer, pH 4.5 (GSTA_{control}). The SAR_{prodrug} is proportional to the amount of GST isozymes induced by the MMF prodrug treatment while the SAR_{DMF} is proportional to the amount of GST isozymes induced by DMF prodrug treatment. A detailed description of how to determine the GSTAᵣₑₗ is provided in Example 1 herein.

In certain embodiments of the presently disclosed methods, the GSTAᵣₑₗ is less than 80%. In other embodiments, the GSTAᵣₑₗ is less than 70%. In other embodiments, the GSTAᵣₑₗ is less than 60%. In other embodiments, the GSTAᵣₑₗ is less than 50%. In other embodiments, the GSTAᵣₑₗ is less than 40%. In other embodiments, the GSTAᵣₑₗ is less than 30%. In still other embodiments, the GSTAᵣₑₗ is less than 20%.

It is noted that prodrugs having the disclosed GSTAᵣₑₗ can be combined without limitation with any other disclosed aspect of the present disclosure.

### Ratio of AUC_{MMF 0-24}: AUC_{Prodrug 0-24}

Another characteristic of the MHF prodrug useful in the presently described methods is good in vivo conversion or metabolism of the MHF prodrug to MHF.

The concentration of MHF in blood after oral administration of an MHF prodrug can be carried out either in patients having the disease to be treated or in healthy subjects. After MHF prodrug administration, blood samples are collected and concentrations of MHF and MHF prodrug can be measured in either whole blood or in blood plasma. By comparing the areas under the time-concentration curves (AUCs) for MMF and the MMF prodrug, the extent of MMF prodrug conversion/metabolism to MMF can be determined. The extent of MMF prodrug conversion/metabolism to MMF is calculated by first determining the area under the time-concentration curve for MMF from time zero to 24 hours (AUC_{MMF 0-24}) and then determining the area under the time-concentration curve for the MMF prodrug from time zero to 24 hours (AUC_{Prodrug 0-24}). In certain embodiments of the present methods, the ratio of AUC_{MMF 0-24}: AUC_{Prodrug 0-24} is at least 3:1. In mathematical terms,

AUC_{MMF 0-24}÷AUC_{Prodrug 0-24}≧3

In other embodiments of the present methods, the ratio of AUC_{MMF 0-24}. AUC_{Prodrug 0-24} is at least 9:1. In mathematical terms,

AUC_{MMF 0-24}÷AUC_{Prodrug 0-24}≧≧9

In still other embodiments of the present methods, the ratio of AUC_{MMF 0-24}: AUC_{Prodrug 0-24} is at least 19:1. In mathematical terms,

AUC_{MMF 0-24}÷AUC_{Prodrug 0-24}≧19

In accordance with certain embodiments, the MHF prodrug has a human in vivo metabolism to MHF over 24 hours sufficient to achieve a ratio of AUC_{MMF 0-24}: AUC_{Prodrug 0-24} of at least 3:1. In other embodiments, the MHF prodrug has a human in vivo metabolism to MHF over 24 hours sufficient to achieve a ratio of AUC_{MMF 0-24}: AUC_{Prodrug 0-24} of at least 5:1. In other embodiments, the MHF prodrug has a human in vivo metabolism to MHF over 24 hours sufficient to achieve a ratio of AUC_{MMF 0-24}: AUC_{Prodrug 0-24} of at least 7:1. In other embodiments, the MHF prodrug has a human in vivo metabolism to MHF over 24 hours sufficient to achieve a ratio of AUC_{MMF 0-24}: AUC_{Prodrug 0-24} of at least 9:1. In other embodiments, the MHF prodrug has a human in vivo metabolism to MHF over 24 hours sufficient to achieve a ratio of AUC_{MMF 0-24}: AUC_{Prodrug 0-24} of at least 11:1. In other embodiments, the MHF prodrug has a human in vivo metabolism to MHF over 24 hours sufficient to achieve a ratio of AUC_{MMF 0-24}: AUC_{Prodrug 0-24} of at least 13:1. In other embodiments, the MHF prodrug has a human in vivo metabolism to MHF over 24 hours sufficient to achieve a ratio of AUC_{MMF 0-24}: AUC_{Prodrug 0-24} of at least 15:1. In other embodiments, the MHF prodrug has a human in vivo metabolism to MHF over 24 hours sufficient to achieve a ratio of AUC_{MMF 0-24}: AUC_{Prodrug 0-24} of at least 17:1. In still other embodiments, the MHF prodrug has a human in vivo metabolism to MHF over 24 hours sufficient to achieve a ratio of AUC_{MMF 0-24}: AUC_{Prodrug 0-24} of at least 19:1.

It is noted that prodrugs having the disclosed ratios of AUC_{MMF 0-24}: AUC_{Prodrug 0-24} can be combined without limitation with any other disclosed aspect of the present disclosure.

### Gastrointestinal Irritation

Another characteristic of the MHF prodrug is low gastrointestinal irritation. The Annamalai-Ma gastrointestinal irritation rat model is predictive of gastrointestinal irritation of MHF prodrugs in humans. This animal model has several common features of other published GI irritation animal models including the Whiteley-Dalrymple model described in Models of Inflammation: Measuring Gastrointestinal Ulceration in the Rat, Pharmacology (1998) 10.2.1-10.2.4; as well as the animal models disclosed in Joseph J. Bertone, DVM, MS, DipACVIM. Prevalence of Gastric Ulcers in Elite, Heavy Use Western Performance Horses, AAEP Proceedings/Vol. 46/2000; and Isbíl Büyükcoskun N., Central Effects of Glucagon-like Peptide-1 on Cold Restraint Stress-induced Gastric Mucosal Lesions, Physiol. Res. 48: 451-455, 1999. The Annamalai-Ma gastrointentinal irritation model is described in detail in Example 2 herein.

In order to assess gastrointestinal irritation using this model, rats are treated orally with either vehicle or the MHF prodrug to be tested (n=10 per group) at 180 mg-equivalents MHF/kg of animal body weight, dosed once per day for 4 days, followed by necropsy and gastrointestinal evaluation at 24 hrs after the final dose. Evans Blue dye is injected IV (tail vein) to visually emphasize any lesions in the gastrointestinal tissue.

In accordance with certain embodiments, the MHF prodrug has an average gastrointestinal irritation score in an Annamalai-Ma gastrointestinal irritation rat model of no more than 3 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 2.5 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 2 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 1.6 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 1.4 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 1.2 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 1.0 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 0.8 after orally administering a solution or suspension of the prodrug to rats at a dose of 180 mg-equivalents of MHF per kg of body weight, dosed once per day over 4 consecutive days.

In accordance with certain embodiments, the MHF prodrug has an average gastrointestinal irritation score in an Annamalai-Ma gastrointestinal irritation rat model of no more than 0.6 after orally administering a solution or suspension of the prodrug to rats at a dose of 180 mg-equivalents of MHF per kg of body weight, dosed once per day over 4 consecutive days.

It is noted that prodrugs having the disclosed gastrointestinal irritation score in an Annamalai-Ma gastrointestinal irritation rat model can be combined without limitation with any other disclosed aspect of the present disclosure.

For the treatment of multiple sclerosis and/or psoriasis, blood plasma concentrations of MHF of at least 0.5 μg/ml during the course of dosing is desired. In other embodiments, blood plasma concentrations of MHF of at least 0.7 μg/ml during the course of dosing is desired. In other embodiments, blood plasma concentrations of MHF of at least 1.2 μg/ml during the course of dosing is desired. In other embodiments, blood plasma concentrations of MHF of at least 1.0 μg/ml during the course of dosing is desired. It is noted that prodrugs having any disclosed blood plasma concentration of MHF can be combined, without limitation, with any other disclosed aspect of the present disclosure.

Similarly, for the treatment of multiple sclerosis and/or psoriasis, an area under a concentration of MHF in blood plasma versus time curve (AUC) of at least 4.0 μg·hr/ml over 24 hours of dosing is desired. In other embodiments, an area under a concentration of MHF in blood plasma versus time curve (AUC) of at least 4.8 μg·hr/ml over 24 hours of dosing is desired. In other embodiments, an area under a concentration of MHF in blood plasma versus time curve (AUC) of at least 6.0 μg·hr/ml over 24 hours of dosing is desired. In other embodiments, an area under a concentration of MHF in blood plasma versus time curve (AUC) of at least 7.0 μg·hr/ml over 24 hours of dosing is desired. In other embodiments, an area under a concentration of MHF in blood plasma versus time curve (AUC) of at least 9.0 μg·hr/ml over 24 hours of dosing is desired. In other embodiments, an area under a concentration of MHF in blood plasma versus time curve (AUC) of at least 10.5 μg·hr/ml over 24 hours of dosing is desired. In still other embodiments, an area under a concentration of MHF in blood plasma versus time curve (AUC) of at least 12.0 μg·hr/ml over 24 hours of dosing is desired. It is noted that prodrugs having any disclosed area under a concentration of MHF in blood plasma versus time curve (AUC) can be combined, without limitation, with any other disclosed aspect of the present disclosure.

### Pharmaceutical Compositions

The present disclosure relates to pharmaceutical compositions comprising a therapeutically effective amount of an MHF prodrug disclosed herein and a pharmaceutically acceptable carrier, (also known as a pharmaceutically acceptable excipient). As discussed above, MHF prodrugs are used for the treatment of multiple sclerosis and psoriasis. Pharmaceutical compositions for the treatment of those diseases and disorders contain a therapeutically effective amount of an MHF prodrug as appropriate for treatment of a patient with the particular disease or disorder.

A “therapeutically effective amount” of an MHF prodrug refers to an amount sufficient to achieve blood plasma concentrations of MHF of at least 0.5 μg/ml during the course of dosing and an area under a concentration of MHF in blood plasma versus time curve (AUC) of at least 4.8 μg·hr/ml over 24 hours of dosing. In other embodiments, a “therapeutically effective amount” of an MHF prodrug refers to an amount sufficient to achieve blood plasma concentrations of MHF of at least 0.7 μg/ml during the course of dosing and an area under a concentration of MHF in blood plasma versus time curve (AUC) of at least 7.0 μg·hr/ml over 24 hours of dosing. In still other embodiments, a “therapeutically effective amount” of an MHF prodrug refers to an amount sufficient to achieve blood plasma concentrations of MHF of at least 1.0 μg/ml during the course of dosing and an area under a concentration of MHF in blood plasma versus time curve (AUC) of at least 12.0 μg·hr/ml over 24 hours of dosing. The actual amount required for treatment of any particular patient will depend upon a variety of factors including the disorder being treated and its severity; the specific pharmaceutical composition employed; the age, body weight, general health, sex and diet of the patient; the dosage form employed; the time of administration; the rate and extent of metabolism of the MHF prodrug to MHF; and the rate of excretion of a disclosed MHF prodrug; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference.

A pharmaceutical composition may be any pharmaceutical form which maintains the MHF prodrug in a stable form and which can be orally administered to a patient. The pharmaceutical composition may be a solid form or a liquid solution or suspension.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition, that is one having a MHF prodrug disclosed herein, a carrier should be chosen that maintains the MHF prodrug in an intact form. In other words, the carrier should not substantially allow premature breakdown of the MHF prodrug into MHF. Nor should the carrier be otherwise incompatible with a MHF prodrug, such as by producing any undesirable biological effect or otherwise interacting in a deleterious manner with any other component(s) of the pharmaceutical composition.

The pharmaceutical compositions are preferably formulated in unit dosage form for ease of administration and uniformity of dosage. A “unit dosage form” refers to a physically discrete unit of therapeutic agent appropriate for the patient to be treated. It will be understood, however, that the total daily dosage of a MHF prodrug and its pharmaceutical compositions will be decided by the attending physician within the scope of sound medical judgment.

Solid dosage forms are a particularly suitable form for the pharmaceutical compositions. Solid dosage forms for oral administration include capsules, tablets, pills, powders, and granules. In such solid dosage forms, the MHF prodrug is mixed with at least one inert, pharmaceutically acceptable carrier. The solid dosage form may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, starch, alginic acid, certain silicates, and sodium carbonate; e) dissolution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate. The solid dosage forms may also comprise buffering agents. They may optionally contain opacifying agents and can also be of a composition that they release the MHF prodrug only, or preferentially, in a certain part of the intestinal tract, optionally, in a delayed manner. For example, the dosage form can be an enteric coated tablet that releases the MHF prodrug after it passes out of the low pH environment of the stomach. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions can also be prepared with coatings and shells, including enteric coatings and other coatings well known in the pharmaceutical formulating art.

An MHF prodrug can be in a solid micro-encapsulated form with one or more carriers as discussed above. Microencapsulated forms of a MHF prodrug may also be used in soft and hard-filled gelatin capsules with carriers such as lactose or other sugars as well as high molecular weight polyethylene glycols and the like.

### Dosing

The multiple sclerosis and psoriasis treatment methods disclosed herein are not limited to any particular oral dosing regimen or any particular oral dosage form, as long as the dosing regimen and dosage form achieves the blood plasma concentration levels and AUC levels described above. The MHF prodrugs may be administered at dosage levels of about 0.001 mg/kg to about 50 mg/kg, from about 0.01 mg/kg to about 25 mg/kg, or from about 0.1 mg/kg to about 10 mg/kg of subject body weight per day, one, two, three, four or more times a day, to obtain the desired concentrations and AUC for MHF in the blood plasma.

### MHF Prodrug Compounds

In certain embodiments, the MHF prodrug is a compound of Formula (I):

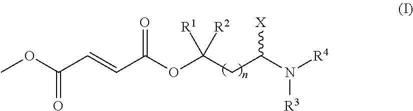

or a pharmaceutically acceptable salt thereof, wherein:

R¹ and R² are independently chosen from hydrogen, C₁₋₆ alkyl, and substituted C₁₋₆ alkyl;

R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₄₋₁₀ heteroaryl, substituted C₄₋₁₀ heteroaryl, C₄₋₁₀ heterocycloalkyl, and substituted C₄₋₁₀ heterocycloalkyl;

n is an integer from 0 to 4; and

X is independently chosen from a single oxygen atom and a pair of hydrogen atoms;

wherein each substituent group is independently chosen from halogen, —OH, —CN, —CF₃, ═O, —NO₂, benzyl, —C(O)NR¹¹₂, —R¹¹, —OR, —C(O)R¹¹, —COOR¹¹, and —NR¹¹₂ wherein each R¹¹ is independently chosen from hydrogen and C₁₋₄ alkyl;

and wherein when X is a single oxygen atom, the oxygen atom is connected to the carbon to which it is bonded by a double bond to form a carboxyl group and when X is a pair of hydrogen atoms, each hydrogen atom is connected to the carbon to which it is bonded to by single bond.

Of the MMF prodrugs of Formula (I), it is important to keep in mind that not all of these prodrugs are suitable for the methods described herein. Rather, it is first necessary to test the relative GST enzyme activity, in vivo metabolism and gastrointestinal irritation scores in accordance with the methods and standards described herein to determine if any particular Formula (I) MHF prodrug is suitable for use in the methods described herein.

Likewise, potentially suitable MHF prodrugs for use in the present methods of treating multiple sclerosis and/or psoriasis are those compounds disclosed in Gangakhedkar et al., U.S. Pat. No. 8,148,414, and specifically the formula (I) compounds therein wherein R⁵ is methyl, the disclosures of which are incorporated herein by reference. The methods and schemes of synthesis in U.S. Pat. No. 8,148,414 are incorporated by reference.

Additional potentially suitable MHF prodrugs for use in the present methods of treating multiple sclerosis and/or psoriasis are those compounds disclosed in Cundy et al., U.S. Patent Application No. 61/595,835 filed Feb. 7, 2012, and specifically the formula (I) compounds therein wherein R¹ is methyl, the disclosures of which are incorporated herein by reference. For example, when considering a particular compound from the Gangakhedkar et al. or Cundy et al. MHF prodrug compounds, it is first necessary to test the relative GST enzyme activity, in vivo metabolism and gastrointestinal irritation scores in accordance with the methods and standards described herein to determine if the particular MHF prodrug is suitable for use in the methods described herein.

The following MHF prodrugs have suitable relative GST enzyme activity, in vivo metabolism and gastrointestinal irritation scores to be used in the presently disclosed treatment methods:

(N,N-Diethylcarbamoyl)methyl methyl (2E)but-2-ene-1,4-dioate,

(N,N-Dimethylcarbamoyl)methyl methyl (2E)but-2-ene-1,4-dioate,

(N-cyclopropyl-N-ethylcarbamoyl)methyl methyl 2(E)but-2-ene-1,4-dioate, and

Methyl 4-morpholin-4-ylbutyl (2E) but-2-ene-1,4-dioate, and pharmaceutically acceptable salts thereof.

### EXAMPLES

The following examples illustrate various aspects of the disclosure. It will be apparent to those skilled in the art that many modifications, both to materials and methods, may be practiced without departing from the scope of the disclosure.

All reagents and solvents that are purchased from commercial suppliers are used without further purification or manipulation procedures.

### General Procedure A: Nucleophilic Substitution of 1-Haloacetamides or 1-Halo Acetic Acid Derivatives with Monomethyl Fumarate

(2E)-3-(Methoxycarbonyl)prop-2-enoic acid (methyl hydrogen fumarate, MHF), (2E)-3-(tert-butoxycarbonyl)prop-2-enoic acid (tert-butyl hydrogen fumarate), or fumaric acid (FA) (1.0 equivalents) is dissolved in 5-10 mL/3.0 mmol of an inert solvent such as N-methyl pyrrolidone (NMP), N,N-dimethylformamide (DMF), N,N-dimethylacetamide (DMA, DMAc), acetonitrile (MeCN), dimethylsulfoxide (DMSO), tetrahydrofuran (THF), toluene, or mixtures thereof. To the solution, 0.8 to 1.2 equivalents of an appropriate inorganic base such as cesium hydrogen carbonate (CsHCO₃), cesium carbonate (Cs₂CO₃), or potassium carbonate (K₂OC₃) is added. Alternatively, 0.8 bis 1.2 equivalents of a silver salt such silver (I) oxide (Ag₂O) or silver (I) carbonate (Ag₂CO₃); an organic secondary or tertiary base such as dicyclohexylamine (DCHA), triethylamine (TEA), diisopropylethylamine (DIEA), tetrabutylammonium hydroxide (TBAOH), amidine; or a guanidine-based base such as 1,5-diazabicyclo[4.3.0]non-5-ene (DBN), 1,8-diazabicyclo[5.4.0]undec-7-ene (DBU), or 1,1,3,3-tetramethylguanidine (TMG), can be employed. The corresponding alkali, silver, di-, tri- and tetraalkylammonium, amidine, or guanide salt of monoalkyl fumarate can also be preformed. The solution is stirred for 10-60 min at room temperature followed by addition of 0.8-1.2 equivalents of an appropriately functionalized 1-haloacetamide, 1-halo acetic acid derivative, acyloxyalkyl halide, alky- or aryloxycarbonyloxyalkyl halide, or halo alkylmorpholine. The reaction mixture is stirred overnight at a temperature between 40 to 100° C. After cooling to room temperature, insolubles can optionally be filtered off and the reaction mixture diluted with one molar (1.0 M) hydrochloric acid (HCl) and an appropriate organic solvent such as methyl tert-butyl ether (MTBE), diethyl ether (Et₂O), ethylacetate (EtOAc), or mixtures thereof. After phase separation, the aqueous phase is extracted several times with the same solvent. The combined organic extracts are washed with water, brine, and dried over anhydrous magnesium sulfate (MgSO₄). After filtration, the organic solvents are removed under reduced pressure using a rotary evaporator. If required, the crude reaction products are further purified by well known purification techniques such as silica gel flash column chromatography (i.e., Biotage), mass-guided reversed-phase preparative HPLC/lyophilization, precipitation, or crystallization.

### Example 1

### Measurement of Glutathione S Transferase (GST) Activity of the Cytosolic Extracts of Rat Forestomach

Treatment of Rats and Isolation of Cytosolic Extract from Rat Forestomach

Rats are treated for 14 days via oral gavage with vehicle or MHF prodrug dosed at 180 mg-equivalents of MMF/kg of body weight, dosed once per day over the 14 days. Animals are sacrificed on day 15 and stomach tissue is dissected into forestomach and glandular portions after removal of food. All tissues are frozen in liquid nitrogen within minutes of death and stored at −80° C. until the enzyme activities can be assayed. Forestomach tissues are homogenized in 0.25 M sucrose (3.0 ml/g of tissue) at 0-4° C. After centrifugation at 5,000×g for 20 min, the supernatant fluid is collected, 0.2 volume of 0.1 M CaCl₂ in 0.25 M sucrose is added to each sample and the samples are maintained on ice for 30 min. Centrifugation at 15,000×g for 20 min yields clear cytosol fractions suitable for enzyme assays.

### Determination of GST Activity of Rat Forestomach Cytosolic Extracts

Total cytosolic GST activity is determined by measuring the conjugation of 1-chloro-2,4-dinitrobenzene (CDNB) with reduced glutathione. (Habig, W. H., et. al. J Biol Chem 1974, 249 7130-7139) A volume of the forestomach cytosolic fraction is adjusted to pH 6.5 (to minimize the non-enzymatic reactions) and is treated with CDNB and glutathione so that each is present in the solution at 1 mM concentration. The formation of the conjugate is monitored spectrophotometrically by measuring the rate of increase in absorbance at 340 nm over a period of 3 minutes. The rate of increase is measured in μmole/min production of GST-CDNB and is directly proportional to the GST activity in the sample.

The relative GST activity between six animals treated with the MMF prodrug (N,N-diethylcarbamoyl)methyl methyl (2E)but-2-ene-1,4-dioate (GSTA_{prodrug}) 1 and GST activity observed from six control animals (GSTA_{control}) is expressed as a specific activity ratio GSTA_{prodrug}:GSTA_{control}. This specific activity ratio is proportional to the amount of GST isozymes induced by the MMF prodrug treatment.

The level of GST enzyme activity induced by the MMF prodrug (N,N-diethylcarbamoyl)methyl methyl (2E)but-2-ene-1,4-dioate is then compared to that induced by DMF treatment by dividing the specific activity ratio of the MMF prodrug (SAR_{prodrug}) by the specific activity ratio of DMF (SAR_{DMF}) to calculate a relative GST enzyme activity (GSTAᵣₑₗ) in accordance with the following equation (I):

GSTAᵣₑₗ(%)=(SAR_{prodrug}÷SAR_{DMF})×100 (I)

For the MHF prodrug (N,N-diethylcarbamoyl)methyl methyl (2E)but-2-ene-1,4-dioate, the GSTAᵣₑₗ is calculated to be less than 80%.

### Example 2

### Gastrointestinal Irritation Evaluation in Rats

Rats were dosed once per day for 4 consecutive days with 180 mg-equivalents MHF/kg body weight per day of a number of MHF prodrugs. The animals were fasted overnight prior to necropsy. On Day 5, to help visualize lesions, 1 mL of 1% Evan's blue in saline was injected into the tail vein 30 minutes prior to euthanasia. All animals were euthanized by inhalation of carbon dioxide. A partial necropsy, limited to the abdominal cavities, was performed. The stomach and small intestine were removed. Residual material was washed away, using an irrigation syringe filled with saline. The stomach was cut along the larger curvature and washed gently with normal saline, and was examined for any lesions. The stomachs were scored in accordance with the scoring system outlined in Table 1. The stomach scores from at least 5 rats were used to calculate an average gastrointestinal irritation score, which scores are reported in Table 2.

TABLE 1
Scoring System for Stomach Lesions in the Rat

| Score | Characteristics |
|---|---|
| 0 | Normal mucosa. |
| 1 | Non-erosive mucosal changes. Swelling and reddening |
| | without any apparent mucosal defect. |
| 2 | Apparent mucosal erosions. |
| 3 | Mild ulceration 1-5 small lesions (1-2 mm). |
| 4 | Moderate ulceration: More than 5 small lesions or 1 |
| | intermediate lesion (3-4 mm). |
| 5 | Severe ulceration: two or more intermediate lesions |
| | or gross lesions (longer than 4 mm). |

TABLE 2
MHF Prodrug Gastrointestinal Irritation Scores

| | GI Irritation |
|---|---|
| | Score |
| | (180 mg-eq |
| MHF Prodrug Compound | of MHF/kg) |
| Methyl 2-morpholin-4-ylethyl (2E)but-2-ene-1,4-dioate, | 0.0 |
| HCl salt | |
| Methyl 3-morpholin-4-ylpropyl (2E)but-2-ene-1,4-dioate, | 0.0 |
| HCl salt | |
| Methyl 6-morpholin-4-ylhexyl (2E)but-2-ene-1,4-dioate, | 0.0 |
| HCl salt | |
| Methyl 5-morpholin-4-ylpentyl (2E)but-2-ene-1,4-dioate, | 0.0 |
| HCl salt | |
| Methyl 4-morpholin-4-ylbutyl (2E)but-2-ene-1,4-dioate, | 0.6 |
| HCl salt | |
| (N-cyclopropyl-N-ethylcarbamoyl)methyl methyl | 1.0 |
| 2(E)but-2-ene-1,4-dioate | |
| (N,N-diethylcarbamoyl)methyl methyl | 1.4 |
| (2E)but-2-ene-1,4-dioate | |
| (N,N-Dimethylcarbamoyl)methyl methyl | 2.4 |
| (2E)but-2-ene-1,4-dioate | |
| (N-cyclopropyl-N-methylcarbamoyl)methyl methyl | 2.6 |
| (2E)but-2-ene-1,4-dioate | |
| Methyl 2-morpholin-4-yl-2-oxoethyl | 2.9 |
| (2E)but-2-ene-1,4-dioate | |
| Methyl 2-oxo-2-pyrrolidinylethyl 2(E)but-2-ene-1,4-dioate | 3.0 |
| Dimethyl Fumarate | 4.6 |

### Example 3

### Measurement of In Vivo MHF Prodrug Conversion to MMF and Related Pharmacokinetic Parameters

After an overnight fasting for at least 10 hours before MHF prodrug administration, blank samples of EDTA-blood are obtained before administration of the test MHF prodrug with 250 ml of water. The first meal is taken no earlier than 4 hours after MHF prodrug oral administration to ensure complete prodrug absorption under fasting conditions. During the sampling period, patients are allowed to drink water only. EDTA-blood samples are obtained every 15 minutes during the first 90 minutes, in 30 minute intervals for the next 150 minutes and then in 60 minute intervals up to 24 hours.

EDTA-blood samples are immediately cooled on ice for 5 minutes before centrifugation (1,717 g, 15 min, 4° C.). Plasma obtained is aliquoted to 500 μl aliquots, which serve for determination of MMF, are mixed with 500 ul of 0.2 M hydrochloric acid (Sigma-Aldrich, Germany) and are stored at −70° C. before solid phase extraction (SPE).

### HPLC Assay for the Determination of MHF Prodrugs and MHF in Plasma

Plasma samples (500 μl plasma mixed with 500 μl 0.2 M HCl) are prepared by a SPE procedure. Strata X-cartridges (30 mg sorbent, Phenomenex, AschaVenburg, Germany) are conditioned with 1 ml methanol (J. T. Baker, Deventer, Holland) and are equilibrated with 1 ml 0.02 M HCl followed by a drying step of 30 sec under vacuum. Thereafter, the complete sample is loaded onto the cartridge. A washing step with 1 ml 0.02 M HCl containing 5% methanol is followed by a second drying for 2 min under vacuum. The analytes are eluted with 500 μl distilled water (J. T. Baker, Deventer, Holland) containing 30% acetonitrile (J. T. Baker, Deventer, Holland). The resulting solution is diluted with distilled water 1:3 (v/v) before 20 μl is injected into the HPLC system. A VWR LaChrom Elite HPLC system (Darmstadt, Germany) with a Hitachi L-2400 UV detector set to 215 nm combined with a Gerstel (Mülheim, Germany) MPS-3 auto sampler is used. Analysis are carried out on a LiChroCARTO 250 £ 4 mm LiChrospher® 60 RP-select B (5 μm) with an appropriate precolumn (Merck, Darmstadt, Germany) at a flow rate of 1.0 ml/min. The column is placed in a column oven and kept at 25° C. All solvents are degassed by a Gilson (Middleton, USA) 864 degasser and are filtered before entering the column. An isocratic system is used. The mobile phases are A: 50 mM phosphate buffer with 25 mM tetrabutylammonium bisulfate (pH=5.5) and B: acetonitrile 80:20 (v/v).

### Determination of Cₘₐₓ and Area Under the Concentration Versus Time Curve of MHF Prodrug and MHF in Blood Plasma

Pure standards of MHF and the MHF prodrugs are diluted for calibration with a mixture of distilled water and acetonitrile [70:30(v/v)]. Similar to experimental samples, they are diluted 1:3 with distilled water before injection in to the HPLC system. Calibration curves for MHF prodrugs and MHF are obtained in a range of 0.04 to 4 μg/ml. To estimate recovery rates blank plasma with defined concentrations of analytes ranging from 0.04 to 4 μg/ml are analyzed.

Pharmacokinetic parameters (Cₘₐₓ and AUC) of MHF prodrugs and MHF are calculated according to a non-compartmental model using the software WinNonlin (Pharsight, Mountain View, Calif.).

### Example 4

### Use of DMF and MHF Prodrug in Animal Models of Multiple Sclerosis and Psoriasis

The following experiment confirmed that MHF is the active moiety of both MHF prodrugs DMF and (N,N-diethylcarbamoyl)methyl methyl (2E)but-2-ene-1,4-dioate and examined the relationship between MHF exposure and effect in animal models of multiple sclerosis (MS) and psoriasis. Efficacy of (N,N-diethylcarbamoyl)methyl methyl (2E)but-2-ene-1,4-dioate and DMF was compared in the MOG35-55 mouse EAE model of multiple sclerosis. C57BL/6 mice (6 females) were injected subcutaneously with MOG35-55 peptide in CFA with Mycobacterium tuberculosis. Pertussis toxin (200 mg) was injected IV on Day 0 and Day 2 post-immunization. Animals received oral XP23829 or DMF (90 mg-eq MHF/kg twice daily) or vehicle on Days 3 to 29. Daily EAE clinical disease scores (5 point scale) were recorded. End of study MHF blood levels were determined by LC/MS/MS.

Efficacy of (N,N-diethylcarbamoyl)methyl methyl (2E)but-2-ene-1,4-dioate and DMF was compared in the imiquimod (IMQ) mouse model of psoriasis. Balb/c mice (10 males/group) received daily topical IMQ (5% cream) on shaved back and right ear for 5 days. Animals received oral (N,N-diethylcarbamoyl)methyl methyl (2E)but-2-ene-1,4-dioate or DMF (45 or 90 mg-eq MMF/kg twice daily) or vehicle from Day −5 to Day 5. Erythema score was the primary outcome measure.

In the EAE Model, (N,N-diethylcarbamoyl)methyl methyl (2E)but-2-ene-1,4-dioate produced significant reduction in EAE clinical score (Day 29 and overall AUC) compared to vehicle. DMF effect was not statistically significant. In the IMQ Model, (N,N-diethylcarbamoyl)methyl methyl (2E)but-2-ene-1,4-dioate effect was significant versus control at 90 mg-eq/kg while DMF effect was not significant at either dose. MHF systemic exposures were approx. 30% higher after dosing (N,N-diethylcarbamoyl)methyl methyl (2E)but-2-ene-1,4-dioate compared to DMF at molar equivalent doses. Intact (N,N-diethylcarbamoyl)methyl methyl (2E)but-2-ene-1,4-dioate and DMF were not detected in the systemic blood.

(N,N-diethylcarbamoyl)methyl methyl (2E)but-2-ene-1,4-dioate demonstrated significantly greater efficacy than DMF at molar equivalent doses in animal models of multiple sclerosis and psoriasis. The substantial absence of circulating prodrug (AUC_{MMF 0-24}: AUC_{Prodrug 0-24} much greater than 20:1) supports the conclusion that MHF is the active moiety of both compounds. The enhanced activity of (N,N-diethylcarbamoyl)methyl methyl (2E)but-2-ene-1,4-dioate may reflect improved absorption and delivery of the active moiety MHF compared to dosing DMF.

### Example 5

### Comparative Gastric Irritation of MHF Prodrug and DMF in Rat and Monkey

The fumaric acid esters (N,N-diethylcarbamoyl)methyl methyl (2E)but-2-ene-1,4-dioate and DMF are MHF prodrugs. The objective of the following experiment was to compare the potential for oral administration of (N,N-diethylcarbamoyl)methyl methyl (2E)but-2-ene-1,4-dioate and DMF to cause prolonged gastrointestinal irritation in animals after repeated dosing.

(N,N-diethylcarbamoyl)methyl methyl (2E)but-2-ene-1,4-dioate was administered to CD rats (10/sex/dose) at 0 (vehicle), 150, 250, or 500 mg/kg/day and monkeys (3/sex/group) at 0 (vehicle), 25, 75, or 200 mg/kg/day for 4 weeks in GLP toxicity studies. In parallel studies, DMF was administered at doses intended to deliver similar MHF exposures: in rats (10/sex/dose) at 90, 150, and 300 mg/kg/day and monkeys (3/sex/group) at 0 (vehicle), 40, 90, or 250 mg/kg/day for 4 weeks. Gastrointestinal toxicity was assessed by gross necropsy and histopathology. Toxicokinetics were assessed at steady state in parallel cohorts. The tissue distribution of 14C-DMF and 14C-XP23829 (labeled in the fumarate moieties) were compared in rats by quantitative whole body autoradiography.

DMF caused dose dependent gastrointestinal irritation in rats and monkeys after 4 weeks of dosing. In rats, DMF at 300 mg/kg/day caused ulceration, necrosis and loss of mucosa of glandular stomach. In contrast, (N,N-diethylcarbamoyl)methyl methyl (2E)but-2-ene-1,4-dioate at 500 mg/kg/day showed no adverse effects on glandular stomach while delivering an identical MHF exposure. One high dose DMF monkey died from gastric ulceration; histopathology showed greater gastric mucosal hyperplasia compared to (N,N-diethylcarbamoyl)methyl methyl (2E)but-2-ene-1,4-dioate. Both DMF and (N,N-diethylcarbamoyl)methyl methyl (2E)but-2-ene-1,4-dioate were converted rapidly to MHF after absorption and levels of released MHF were similar. DMF and (N,N-diethylcarbamoyl)methyl methyl (2E)but-2-ene-1,4-dioate were not detected in the systemic circulation of either species. Radioactivity in stomach mucosa was 4.5 fold higher for 14C-DMF than for 14C-(N,N-diethylcarbamoyl)methyl methyl (2E)but-2-ene-1,4-dioate at 0.25 hr. Concentrations in other tissues were similar for both compounds.

At similar systemic exposures to the active moiety MHF, DMF accumulated in rat stomach more than (N,N-diethylcarbamoyl)methyl methyl (2E)but-2-ene-1,4-dioate and caused significantly greater local gastric irritation after 4 weeks dosing in rats and monkeys. (N,N-diethylcarbamoyl)methyl methyl (2E)but-2-ene-1,4-dioate may have the potential for a lower risk of gastrointestinal side effects in clinical use compared to DMF.

### Example 6

### (N,N-Diethylcarbamoyl)methyl methyl (2E)but-2-ene-1,4-dioate

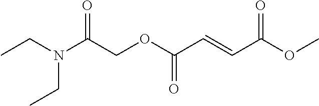 [SMILES: CCN(CC)C(=O)COC(=O)/C=C/C(=O)OC]

Following general procedure A, methyl hydrogen fumarate (MHF) (0.39 g, 3.00 mmol) dissolved in NMP was reacted at about 55° C. with 2-chloro-N,N-diethylacetamide (0.44 g, 3.00 mmol) in the presence of CsHCO₃ (0.69 g, 3.60 mmol) to afford 0.37 g (51% yield) of the title compound after purification by silica gel column chromatography (Biotage) using a mixture of ethyl acetate (EtOAc) and hexanes (1:1) as eluent. M.p.: 53-56° C. ¹H NMR (CDCl₃, 400 MHz): δ 6.99-6.90 (m, 2H), 4.83 (s, 2H), 3.80 (s, 3H), 3.39 (q, J=7.2 Hz, 2H), 3.26 (q, J=7.2 Hz, 2H), 1.24 (t, J=7.2 Hz, 3H), 1.14 (t, J=7.2 Hz, 3H). MS (ESI): m/z 244.13 (M+H)⁺.

### Example 7

### Methyl 2-morpholin-4-yl-2-oxoethyl (2E)but-2-ene-1,4-dioate

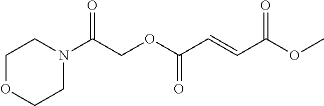 [SMILES: COC(=O)/C=C/C(=O)OCC(=O)N1CCOCC1]

Following general procedure A, methyl hydrogen fumarate (MHF) (0.50 g, 3.84 mmol) dissolved in NMP was reacted at about 55° C. with 4-(chloroacetyl) morpholine (0.75 g, 4.61 mmol) in the presence of CsHCO₃ (0.89 g, 4.61 mmol) to afford 0.34 g (35% yield) of the title compound as a white solid after purification by mass-guided preparative HPLC and lyophilization. M.p.: 124 to 126° C.; ¹H NMR (CDCl₃, 400 MHz): δ 6.97-6.91 (m, 2H), 4.84 (s, 2H), 3.82 (s, 3H), 3.72-3.70 (m, 4H), 3.64-3.62 (m, 2H), 3.46-3.41 (m, 2H). MS (ESI): m/z 258.04 (M+H)⁺.

### Example 8

### N,N-Dimethylcarbamoyl)methyl methyl (2E)but-2-ene-1,4-dioate

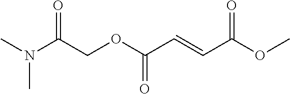

Following general procedure A, methyl hydrogen fumarate (MHF) (0.50 g, 3.84 mmol) dissolved in NMP was reacted at about 55° C. with N,N-dimethyl chloroacetamide (0.56 g, 4.61 mmol) in the presence of CsHCO₃ (0.89 g, 4.61 mmol). The crude material was precipitated out from a mixture of ethyl acetate (EtOAc) and hexanes (Hxn) (1:1) to provide a white solid. This solid was further dissolved in dichloromethane (DCM) and the organic layer washed with water. After removal of the solvents 0.55 g (67% yield) of the title compound was obtained as a white solid. ¹H NMR (CDCl₃, 400 MHz): δ 6.98-6.90 (m, 2H), 4.84 (s, 2H), 3.80 (s, 3H), 2.99-2.97 (2s, 6H). MS (ESI): m/z 216 (M+H)+.

### Example 9

### Methyl (2-morpholino-4-ylethyl) fumarate

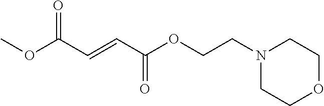 [SMILES: COC(=O)/C=C/C(=O)OCCN1CCOCC1]

Following general Procedure A, methyl hydrogen fumarate (MHF) dissolved in NMP is reacted at about 55° C. with 4-(chloroethyl) morpholine (0.75 g, 4.61 mmol) in the presence of CsHCO₃ to afford the title compound after purification by mass-guided preparative HPLC and lyophilization.

### Example 10

### Methyl (3-morpholino-4-ylpropyl) fumarate

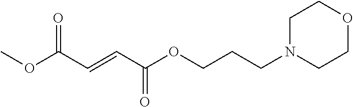 [SMILES: COC(=O)/C=C/C(=O)OCCCN1CCOCC1]

Following the procedure of Methyl (2-morpholino-4-ylethyl) fumarate, and replacing 4-(chloroethyl) morpholine with 4-(chloropropyl) morpholine provides the title compound.

### Example 11

### Methyl (4-morpholino-4-ylbutyl) fumarate

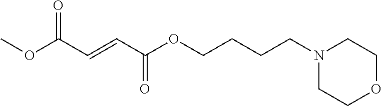 [SMILES: COC(=O)/C=C/C(=O)OCCCCN1CCOCC1]

Following the procedure of Methyl (2-morpholino-4-ylethyl) fumarate, and replacing 4-(chloroethyl) morpholine with 4-(chlorobutyl) morpholine provides the title compound.

### Example 12

### Methyl (5-morpholino-4-ylpentyl) fumarate

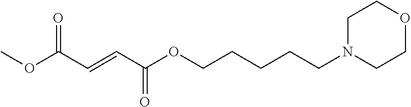 [SMILES: COC(=O)/C=C/C(=O)OCCCCCN1CCOCC1]

Following the procedure of Methyl (2-morpholino-4-ylethyl) fumarate, and replacing 4-(chloroethyl) morpholine with 4-(chloropentyl) morpholine provides the title compound.

### Example 13

### (N-cyclopropyl-N-ethylcarbamoyl)methyl methyl 2(E)but-2-ene-1,4-dioate

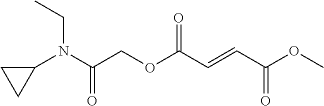 [SMILES: CCN(C(=O)COC(=O)/C=C/C(=O)OC)C1CC1]

Following the general procedure A, methyl hydrogen fumarate (MHF) (38.7 g, 0.297 mol) suspended in toluene (100 mL) was reacted at about 80° C. with 2-chloro-N-cyclopropyl-N-ethylacetamide (48 g, 0.297 mol) in the presence of N,N-diisopropylethylamine (DIEA; 42.3 g, 57 mL, 0.327 mol) to afford 50 g (63.3%) of the title compound after recrystallization using methyl tert-butyl ether. The crystalline compound had a melting point of 92.1° C. ¹H NMR (CDCl₃, 400 MHz): δ 7.01-6.92 (m, 2H), 4.99 (s, 2H), 3.81 (s, 3H), 3.44 (q, J=7.2 Hz, 2H), 2.69-2.66 (m, 1H), 1.14 (t, J=7.2 Hz, 3H), 0.94-0.91 (m, 2H), 0.83-0.81 (m, 2H). MS (ESI): m/z 256.2 (M+H)⁺.

### Example 14

### (N-cyclopropyl-N-methylcarbamoyl)methyl methyl 2(E)but-2-ene-1,4-dioate

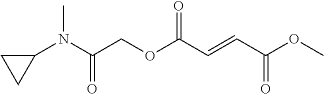 [SMILES: COC(=O)/C=C/C(=O)OCC(=O)N(C)C1CC1]

Following general procedure A, methyl hydrogen fumarate (MHF) (38.7 g, 0.40 mol) suspended in toluene (100 mL) was reacted at about 80° C. with 2-chloro-N-cyclopropyl-N-methylacetamide (60 g, 0.40 mol) in the presence of N,N-diisopropylethylamine (DIEA; 57.8 g, 78 mL, 0.44 mol) to afford 50 g (50.86%) of the title compound after recrystallization using methyl tert-butyl ether. The crystalline compound had a melting point of 93.6° C. ¹H NMR (CDCl₃, 400 MHz): δ 7.01-6.91 (m, 2H), 5.01 (s, 2H), 3.82 (s, 3H), 2.94 (s, 3H), 2.73-2.68 (m, 1H), 0.94-0.86 (m, 2H), 0.83-0.78 (m, 2H). MS (ESI): m/z 242.2 (M+H)⁺.

### Example 15

### Methyl 2-oxo-2-pyrrolidinylethyl 2(E)but-2-ene-1,4-dioate

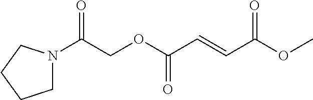 [SMILES: COC(=O)/C=C/C(=O)OCC(=O)N1CCCC1]

Following general procedure A, methyl hydrogen fumarate (MHF) (20.78 g, 0.159 mol) suspended in toluene (60 mL) was reacted at about 80° C. with 2-chloro-1-pyrrolidin-1-yl-ethanone (23.5 g, 0.159 mol) in the presence of N,N-diisopropylethylamine (DIEA; 22.69 g, 31.5 mL, 0.175 mol) to afford 24 g (62.3%) of the title compound after recrystallization using methyl tert-butyl ether. The crystalline compound had a melting point of 102.1° C. ¹H NMR (CDCl₃, 400 MHz): δ 7.00-6.92 (m, 2H), 4.75 (s, 2H), 3.81 (s, 3H), 3.53-3.49 (t, J=6.8 Hz, 2H), 3.42-3.39 (t, J=6.8 Hz, 2H), 2.20-1.97 (m, 2H), 1.91-1.82 (m, 2H). MS (ESI): m/z 242 (M+H)⁺.

It should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

1. A compound of Formula (I): 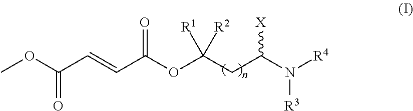
or a pharmaceutically acceptable salt thereof, wherein:
R¹ and R² are each hydrogen;
R³ and R⁴ together with the nitrogen to which they are bonded form a ring chosen from C₄₋₁₀ heterocycloalkyl and substituted C₄₋₁₀ heterocycloalkyl;
n is 0; and
X is pair of hydrogen atoms, wherein each hydrogen atom is connected to the carbon to which it is bonded by a single bond;
wherein each substituent group is ═O.

2. The compound of claim 1, wherein R³ and R⁴ together with the nitrogen to which they are bonded form a four to ten membered heterocycloalkyl ring.

3. The compound of claim 2, wherein R³ and R⁴ together with the nitrogen to which they are bonded form a four to six membered heterocycloalkyl ring.

4. The compound of claim 3, wherein R³ and R⁴ together with the nitrogen to which they are bonded form a six membered heterocycloalkyl ring.

5. The compound of claim 1, wherein R³ and R⁴ together with the nitrogen to which they are bonded form a substituted four to six membered heterocycloalkyl ring.

6. The compound of claim 5, wherein R³ and R⁴ together with the nitrogen to which they are bonded form a substituted five membered heterocycloalkyl ring.

7. A pharmaceutical composition, comprising a therapeutically effective amount of the compound of claim 1, and a pharmaceutically acceptable vehicle.

8. The pharmaceutical composition of claim 1, which is an oral formulation.

9. The pharmaceutical composition of claim 1, wherein the composition comprises a therapeutically effective amount of the compound for the treatment of multiple sclerosis.

10. A pharmaceutical composition, comprising therapeutically effective amount of the compound of claim 4, and a pharmaceutically acceptable vehicle.

11. The pharmaceutical composition of claim 4, which is an oral formulation.

12. The pharmaceutical composition of claim 4, wherein the composition comprises a therapeutically effective amount of the compound for the treatment of multiple sclerosis.

13. A pharmaceutical composition, comprising a therapeutically effective amount of the compound of claim 6, and a pharmaceutically acceptable vehicle.

14. The pharmaceutical composition of claim 6, which is an oral formulation.

15. The pharmaceutical composition of claim 6, wherein the composition comprises a therapeutically effective amount of the compound for the treatment of multiple sclerosis.
